SEQUENCE LISTING (146 sequences)

SEQ ID NO: 1 - DNA, 6 bp, Artificial Sequence
  POL III terminator sequence
tttttt

SEQ ID NO: 2 - DNA, 21 bp, Homo sapiens
aaccaagagcggagcaacgaa

SEQ ID NO: 3 - DNA, 21 bp, Homo sapiens
aattcgttgctccgctcttgg

SEQ ID NO: 4 - DNA, 21 bp, Homo sapiens
aaccaagagcggagcaacgaa

SEQ ID NO: 5 - DNA, 21 bp, Homo sapiens
aattcgttgctccgctcttgg

SEQ ID NO: 6 - DNA, 21 bp, Homo sapiens
aaccagtacgtccacatttcc

SEQ ID NO: 7 - DNA, 21 bp, Homo sapiens
aaggaaatgtggacgtactgg

SEQ ID NO: 8 - DNA, 21 bp, Mus musculus
  misc_feature (1)..(21): Starting position within mouse BACE1 cDNA (Genbank Accession NM_011792) and corresponding human sequences. DNA sequence corresponding to the therapeutic siRNA starting at base 0803.The two 5′ nucleotides AA are optional in MB0803
aagggtgtgtatgtgccctac

SEQ ID NO: 9 - DNA, 21 bp, Mus musculus
  misc_feature (1)..(21): Starting position within mouse BACE1 cDNA (Genbank Accession NM_011792) and corresponding human sequences. DNA sequence corresponding to the therapeutic siRNA starting at base 1663. The two 5′ nucleotides AA are optional in MB1663.
aattggctttgctgtcagcgc

SEQ ID NO: 10 - DNA, 21 bp, Mus musculus
  misc_feature (1)..(21): tarting position within mouse BACE1 cDNA (Genbank Accession NM_011792) and corresponding human sequences. DNA sequence corresponding to the therapeutic siRNA starting at base 1749. The two 5′ nucleotides AA are optional in MB1749.
aagactgtggctacaacattc

SEQ ID NO: 11 - DNA, 21 bp, Mus musculus
  misc_feature (1)..(21): Starting position within mouse BACE1 cDNA (Genbank Accession NM_011792) and corresponding human sequences. DNA sequence corresponding to the therapeutic siRNA starting at base 3249. The two 5′ nucleotides AA are optional in MB3249.
aaggctgcctggagaaaggat

SEQ ID NO: 12 - DNA, 21 bp, Mus musculus
  misc_feature (1)..(21): Starting position within mouse BACE1 cDNA (Genbank Accession NM_011792) and corresponding human sequences. DNA sequence corresponding to the therapeutic siRNA starting at base 0916. The two 5′ nucleotides CA are optional in DhMB0918.
cactgaatcggacaagttctt

SEQ ID NO: 13 - DNA, 21 bp, Mus musculus
  misc_feature (1)..(21): Starting position within mouse BACE1 cDNA (Genbank Accession NM_011792) and corresponding human sequences. DNA sequence corresponding to the therapeutic siRNA starting at base 1129. The two 5′ nucleotides CA are optional in DhMB1131.
catgatcattggtggtatcga

SEQ ID NO: 14 - DNA, 21 bp, Mus musculus
  misc_feature (1)..(21): Starting position within mouse BACE1 cDNA (Genbank Accession NM_011792) and corresponding human sequences. DNA sequence corresponding to the therapeutic siRNA starting at base 1231. The two 5′ nucleotides AA are optional in DhMB1233.
aatcaatggtcaagatctcaa

SEQ ID NO: 15 - DNA, 21 bp, Mus musculus
  misc_feature (1)..(21): Starting position within mouse BACE1 cDNA (Genbank Accession NM_011792) and corresponding human sequences. DNA sequence corresponding to the therapeutic siRNA starting at base 1507. The two 5′ nucleotides CA are optional in DhMB1509.
catccttcctcagcaatacct

SEQ ID NO: 16 - DNA, 21 bp, Mus musculus
  misc_feature (1)..(21): Starting position within mouse BACE1 cDNA (Genbank Accession NM_011792) and corresponding human sequences. DNA sequence corresponding to the therapeutic siRNA starting at base 0683. The two 5′ nucleotides CA are optional in SEC0683.
cagacgctcaacatcctggtg

SEQ ID NO: 17 - DNA, 21 bp, Mus musculus
  misc_feature (1)..(21): Starting position within mouse BACE1 cDNA (Genbank Accession NM_011792) and corresponding human sequences. DNA sequence corresponding to the therapeutic siRNA starting at base 1722. The two 5′ nucleotides AA are optional in SEC1722.
aaggtccgtttgttacggcag

SEQ ID NO: 18 - DNA, 21 bp, Mus musculus
  misc_feature (1)..(21): Starting position within mouse BACE1 cDNA (Genbank Accession NM_011792) and corresponding human sequences. DNA sequence corresponding to the therapeutic siRNA starting at base 2163. The two 5′ nucleotides AA are optional in SEC2163.
aatatccttagacaccacaaa

SEQ ID NO: 19 - DNA, 21 bp, Mus musculus
  misc_feature (1)..(21): Starting position within mouse BACE1 cDNA (Genbank Accession NM_011792) and corresponding human sequences. DNA sequence corresponding to the therapeutic siRNA starting at base 2466. The two 5′ nucleotides AA are optional in SEC2466.
aaacaagaacctatgcgatgc

SEQ ID NO: 20 - DNA, 21 bp, Mus musculus
  misc_feature (1)..(21): Starting position within mouse BACE1 cDNA (Genbank Accession NM_011792) and corresponding human sequences. DNA sequence corresponding to the therapeutic siRNA starting at base 2473. The two 5′ nucleotides AA are optional in SEC2473.
aacctatgcgatgcgaatgtt

SEQ ID NO: 21 - DNA, 22 bp, Mus musculus
  misc_feature (1)..(22): Oligonucleotide MB1749A to construct the DNA encoding for a hairpin loop of RNA corresponding to MB1749.
gaagactgtggctacaacattc

SEQ ID NO: 22 - DNA, 38 bp, Mus musculus
  misc_feature (1)..(38): Oligonucleotide MB1749B to construct the DNA encoding for a hairpin loop of RNA corresponding to MB1749.
ttcaagagagaatgttgtagccacagtcttcttttttg

SEQ ID NO: 23 - DNA, 35 bp, Mus musculus
  misc_feature (1)..(35): Oligonucleotide MB1749C to construct the DNA encoding for a hairpin loop of RNA corresponding to MB1749.
tctcttgaagaatgttgtagccacagtcttcggcc

SEQ ID NO: 24 - DNA, 33 bp, Mus musculus
  misc_feature (1)..(33): Oligonucleotide MB1749D to construct the DNA encoding for a hairpin loop of RNA corresponding to MB1749.
aattcaaaaaagaagactgtggctacaacattc

SEQ ID NO: 25 - DNA, 21 bp, Homo sapiens
  misc_feature (1)..(21): Oligonucleotide MD0188 to construct the DNA encoding for siRNA starting at position 0188 within human Huntington cDNA(Genbank Accession NM_002111.3. The first two 5′ nucleotides AA are optional in MD0188.
aagatggacggccgctcaggt

SEQ ID NO: 26 - DNA, 21 bp, Homo sapiens
  misc_feature (1)..(21): Oligonucleotide MD0358 to construct the DNA encoding for siRNA starting at position 0358 within human Huntington cDNA(Genbank Accession NM_002111.3. The first two 5′ nucleotides AA are optional in MD0358.
aagtccttccagcagcagcag

SEQ ID NO: 27 - DNA, 21 bp, Homo sapiens
  misc_feature (1)..(21): Oligonucleotide MD0813 to construct the DNA encoding for siRNA starting at position 0813 within human Huntington cDNA (Genbank Accession NM_002111.3.). The first two 5′ nucleotides AA are optional in MD0813.
aaggttacagctcgagctcta

SEQ ID NO: 28 - DNA, 21 bp, Homo sapiens
  misc_feature (1)..(21): Oligonucleotide M1066 to construct the DNA encoding for siRNA starting at position 1066 within human Huntington cDNA (Genbank Accession NM_002111.3.). The two 5′ nucleotides AA are optional in M1066.
aaggttttgttaaaggccttc

SEQ ID NO: 29 - DNA, 21 bp, Homo sapiens
  misc_feature (1)..(21): Oligonucleotide M1639 to construct the DNA encoding for siRNA starting at position 1639 within human Huntington cDNA (Genbank Accession NM_002111.3.).The two 5′ nucleotides AA are optional in M1639.
aaaggcaaagtgctcttagga

SEQ ID NO: 30 - DNA, 21 bp, Homo sapiens
  misc_feature (1)..(21): Oligonucleotide M2060 to construct the DNA encoding for siRNA starting at position 2060 within human Huntington cDNA (Genbank Accession NM_002111.3.). The two 5′ nucleotides AA are optional in M2060.
aaattgtgttagacggtaccg

SEQ ID NO: 31 - DNA, 21 bp, Homo sapiens
  misc_feature (1)..(21): Oligonucleotide M2714 to construct the DNA encoding for siRNA starting at position 2714 within human Huntington cDNA (Genbank Accession NM_002111.3.).The two 5′ nucleotides CA are optional in M2714.
caggaaatacattttctttgg

SEQ ID NO: 32 - RNA, 19 nt, Homo sapiens
aguggaugagggagcaggc

SEQ ID NO: 33 - RNA, 19 nt, Homo sapiens
gccugcucccucauccacu

SEQ ID NO: 34 - RNA, 19 nt, Homo sapiens
gcacacaguggaugaggga

SEQ ID NO: 35 - RNA, 19 nt, Homo sapiens
ucccucauccacugugugc

SEQ ID NO: 36 - RNA, 19 nt, Homo sapiens
ugaagugcacacaguggau

SEQ ID NO: 37 - RNA, 19 nt, Homo sapiens
auccacugugugcacuuca

SEQ ID NO: 38 - RNA, 19 nt, Homo sapiens
cacacaguggaugagggag

SEQ ID NO: 39 - RNA, 19 nt, Homo sapiens
cucccucauccacugugug

SEQ ID NO: 40 - RNA, 19 nt, Homo sapiens
gcacacaguagaugaggga

SEQ ID NO: 41 - RNA, 19 nt, Homo sapiens
ucccucaucuacugugugc

SEQ ID NO: 42 - RNA, 19 nt, Homo sapiens
ugaagugcacacaguagau

SEQ ID NO: 43 - RNA, 19 nt, Homo sapiens
aucuacugugugcacuuca

SEQ ID NO: 44 - RNA, 19 nt, Homo sapiens
aguagaugagggagcaggc

SEQ ID NO: 45 - RNA, 19 nt, Homo sapiens
gccugcucccucaucuacu

SEQ ID NO: 46 - RNA, 19 nt, Homo sapiens
cacacaguagaugagggag

SEQ ID NO: 47 - RNA, 19 nt, Homo sapiens
cucccucaucuacugugug

SEQ ID NO: 48 - RNA, 19 nt, Homo sapiens
ggcgcagacuuccaaaggc

SEQ ID NO: 49 - RNA, 19 nt, Homo sapiens
gccuuuggaagucugcgcc

SEQ ID NO: 50 - RNA, 19 nt, Homo sapiens
cacaagggcgcagacuucc

SEQ ID NO: 51 - RNA, 19 nt, Homo sapiens
ggaagucugcgcccuugug

SEQ ID NO: 52 - RNA, 19 nt, Homo sapiens
gcagggcacaagggcgcag

SEQ ID NO: 53 - RNA, 19 nt, Homo sapiens
cugcgcccuugugcccugc

SEQ ID NO: 54 - RNA, 19 nt, Homo sapiens
acaagggcgcagacuucca

SEQ ID NO: 55 - RNA, 19 nt, Homo sapiens
uggaagucugcgcccuugu

SEQ ID NO: 56 - RNA, 19 nt, Homo sapiens
cacaagggcacagacuucc

SEQ ID NO: 57 - RNA, 19 nt, Homo sapiens
ggaagucugugcccuugug

SEQ ID NO: 58 - RNA, 19 nt, Homo sapiens
gcagggcacaagggcacag

SEQ ID NO: 59 - RNA, 19 nt, Homo sapiens
cugugcccuugugcccugc

SEQ ID NO: 60 - RNA, 19 nt, Homo sapiens
ggcacagacuuccaaaggc

SEQ ID NO: 61 - RNA, 19 nt, Homo sapiens
gccuuuggaagucugugcc

SEQ ID NO: 62 - RNA, 19 nt, Homo sapiens
acaagggcacagacuucca

SEQ ID NO: 63 - RNA, 19 nt, Homo sapiens
uggaagucugugcccuugu

SEQ ID NO: 64 - RNA, 19 nt, Homo sapiens
caaugguacagcucuuccu

SEQ ID NO: 65 - RNA, 19 nt, Homo sapiens
aggaagagcuguaccauug

SEQ ID NO: 66 - RNA, 19 nt, Homo sapiens
caucccaaugguacagcuc

SEQ ID NO: 67 - RNA, 19 nt, Homo sapiens
gagcuguaccauugggaug

SEQ ID NO: 68 - RNA, 19 nt, Homo sapiens
ccaucccaaugguacagcu

SEQ ID NO: 69 - RNA, 19 nt, Homo sapiens
agcuguaccauugggaugg

SEQ ID NO: 70 - RNA, 19 nt, Homo sapiens
uuguggccaucccaauggu

SEQ ID NO: 71 - RNA, 19 nt, Homo sapiens
accauugggauggccacaa

SEQ ID NO: 72 - RNA, 19 nt, Homo sapiens
caacgguacagcucuuccu

SEQ ID NO: 73 - RNA, 19 nt, Homo sapiens
aggaagagcuguaccguug

SEQ ID NO: 74 - RNA, 19 nt, Homo sapiens
caucccaacgguacagcuc

SEQ ID NO: 75 - RNA, 19 nt, Homo sapiens
gagcuguaccguugggaug

SEQ ID NO: 76 - RNA, 19 nt, Homo sapiens
ccaucccaacgguacagcu

SEQ ID NO: 77 - RNA, 19 nt, Homo sapiens
agcuguaccguugggaugg

SEQ ID NO: 78 - RNA, 19 nt, Homo sapiens
uuguggccaucccaacggu

SEQ ID NO: 79 - RNA, 19 nt, Homo sapiens
accguugggauggccacaa

SEQ ID NO: 80 - RNA, 19 nt, Homo sapiens
gucggcaagcagagcuccc

SEQ ID NO: 81 - RNA, 19 nt, Homo sapiens
gggagcucugcuugccgac

SEQ ID NO: 82 - RNA, 19 nt, Homo sapiens
agccagucggcaagcagag

SEQ ID NO: 83 - RNA, 19 nt, Homo sapiens
cucugcuugccgacuggcu

SEQ ID NO: 84 - RNA, 21 nt, Homo sapiens
cagccagucggcaagcagagc

SEQ ID NO: 85 - RNA, 21 nt, Homo sapiens
gcucugcuugccgacuggcug

SEQ ID NO: 86 - RNA, 19 nt, Homo sapiens
gucucacagccagucggca

SEQ ID NO: 87 - RNA, 19 nt, Homo sapiens
ugccgacuggcugugagac

SEQ ID NO: 88 - RNA, 19 nt, Homo sapiens
gucagcaagcagagcuccc

SEQ ID NO: 89 - RNA, 19 nt, Homo sapiens
gggagcucugcuugcugac

SEQ ID NO: 90 - RNA, 19 nt, Homo sapiens
agccagucagcaagcagag

SEQ ID NO: 91 - RNA, 19 nt, Homo sapiens
cucugcuugcugacuggcu

SEQ ID NO: 92 - RNA, 21 nt, Homo sapiens
cagccagucagcaagcagagc

SEQ ID NO: 93 - RNA, 21 nt, Homo sapiens
gcucugcuugcugacuggcug

SEQ ID NO: 94 - RNA, 19 nt, Homo sapiens
gucucacagccagucagca

SEQ ID NO: 95 - RNA, 19 nt, Homo sapiens
ugcugacuggcugugagac

SEQ ID NO: 96 - RNA, 19 nt, Homo sapiens
cacauacauuagcucaaac

SEQ ID NO: 97 - RNA, 19 nt, Homo sapiens
guuugagcuaauguaugug

SEQ ID NO: 98 - RNA, 19 nt, Homo sapiens
cagcgucacauacauuagc

SEQ ID NO: 99 - RNA, 19 nt, Homo sapiens
gcuaauguaugugacgcug

SEQ ID NO: 100 - RNA, 19 nt, Homo sapiens
acauacauuagcucaaacu

SEQ ID NO: 101 - RNA, 19 nt, Homo sapiens
aguuugagcuaauguaugu

SEQ ID NO: 102 - RNA, 19 nt, Homo sapiens
cauuagcucaaacugguug

SEQ ID NO: 103 - RNA, 19 nt, Homo sapiens
caaccaguuugagcuaaug

SEQ ID NO: 104 - RNA, 19 nt, Homo sapiens
cacauacaucagcucaaac

SEQ ID NO: 105 - RNA, 19 nt, Homo sapiens
guuugagcugauguaugug

SEQ ID NO: 106 - RNA, 19 nt, Homo sapiens
cagcgucacauacaucagc

SEQ ID NO: 107 - RNA, 19 nt, Homo sapiens
gcugauguaugugacgcug

SEQ ID NO: 108 - RNA, 19 nt, Homo sapiens
acauacaucagcucaaacu

SEQ ID NO: 109 - RNA, 19 nt, Homo sapiens
aguuugagcugauguaugu

SEQ ID NO: 110 - RNA, 19 nt, Homo sapiens
caucagcucaaacugguug

SEQ ID NO: 111 - RNA, 19 nt, Homo sapiens
caaccaguuugagcugaug

SEQ ID NO: 112 - RNA, 19 nt, Homo sapiens
caacaucaaagcaucuuga

SEQ ID NO: 113 - RNA, 19 nt, Homo sapiens
ucaagaugcuuugauguug

SEQ ID NO: 114 - RNA, 19 nt, Homo sapiens
ccggccaacaucaaagcau

SEQ ID NO: 115 - RNA, 19 nt, Homo sapiens
augcuuugauguuggccgg

SEQ ID NO: 116 - RNA, 19 nt, Homo sapiens
uccggccaacaucaaagca

SEQ ID NO: 117 - RNA, 19 nt, Homo sapiens
ugcuuugauguuggccgga

SEQ ID NO: 118 - RNA, 19 nt, Homo sapiens
caaguuuccggccaacauc

SEQ ID NO: 119 - RNA, 19 nt, Homo sapiens
gauguuggccggaaacuug

SEQ ID NO: 120 - RNA, 19 nt, Homo sapiens
caaaaucaaagcaucuuga

SEQ ID NO: 121 - RNA, 19 nt, Homo sapiens
ucaagaugcuuugauuuug

SEQ ID NO: 122 - RNA, 19 nt, Homo sapiens
ccggccaaaaucaaagcau

SEQ ID NO: 123 - RNA, 19 nt, Homo sapiens
augcuuugauuuuggccgg

SEQ ID NO: 124 - RNA, 19 nt, Homo sapiens
uccggccaaaaucaaagca

SEQ ID NO: 125 - RNA, 19 nt, Homo sapiens
ugcuuugauuuuggccgga

SEQ ID NO: 126 - RNA, 19 nt, Homo sapiens
caaguuuccggccaaaauc

SEQ ID NO: 127 - RNA, 19 nt, Homo sapiens
gauuuuggccggaaacuug

SEQ ID NO: 128 - RNA, 19 nt, Homo sapiens
gaauuacuguccccaucuc

SEQ ID NO: 129 - RNA, 19 nt, Homo sapiens
gagauggggacaguaauuc

SEQ ID NO: 130 - RNA, 19 nt, Homo sapiens
gcguugaauuacugucccc

SEQ ID NO: 131 - RNA, 19 nt, Homo sapiens
ggggacaguaauucaacgc

SEQ ID NO: 132 - RNA, 19 nt, Homo sapiens
agcguugaauuacuguccc

SEQ ID NO: 133 - RNA, 19 nt, Homo sapiens
gggacaguaauucaacgcu

SEQ ID NO: 134 - RNA, 19 nt, Homo sapiens
ucuucuagcguugaauuac

SEQ ID NO: 135 - RNA, 19 nt, Homo sapiens
guaauucaacgcuagaaga

SEQ ID NO: 136 - RNA, 19 nt, Homo sapiens
gaaguacuguccccaucuc

SEQ ID NO: 137 - RNA, 19 nt, Homo sapiens
gagauggggacaguacuuc

SEQ ID NO: 138 - RNA, 19 nt, Homo sapiens
gcguugaaguacugucccc

SEQ ID NO: 139 - RNA, 19 nt, Homo sapiens
ggggacaguacuucaacgc

SEQ ID NO: 140 - RNA, 19 nt, Homo sapiens
agcguugaaguacuguccc

SEQ ID NO: 141 - RNA, 19 nt, Homo sapiens
gggacaguacuucaacgcu

SEQ ID NO: 142 - RNA, 19 nt, Homo sapiens
ucuucuagcguugaaguac

SEQ ID NO: 143 - RNA, 19 nt, Homo sapiens
guacuucaacgcuagaaga

SEQ ID NO: 144 - DNA, 20 bp, Artificial Sequence
  PRIMER
ctcgcctccagcatgaaagt

SEQ ID NO: 145 - DNA, 19 bp, Homo sapiens
ggagtattgtggaacttat

SEQ ID NO: 146 - DNA, 19 bp, Homo sapiens
tgacagcagtgttgataaa

(19) United States
(12) Patent Application Publication
(10) Pub. No.: US 2011/0052666 A1
(43) Pub. Date: Mar. 3, 2011

(54) Compositions, Methods, and Systems for SIRNA Delivery

(75) Inventors: William F. Kaemmerer, Edina, MN (US); Michael D. Kaytor, Maplewood, MN (US); Marcy R. Weatherspoon, Inver Grove Heights, MN (US)

(73) Assignee: Medtronic, Inc., Minneapolis, MN (US)

(21) Appl. No.: 12/467,720

(22) Filed: Sep. 3, 2009

(51) Int. Cl. A61K 31/7105; A61M 25/098; A61M 5/142; C07H 21/02; A61K 9/127; A61P 25/28

(52) U.S. Cl. 424/450; 604/529; 604/151; 536/24.5; 514/44 A; 977/700; 977/907

(57) ABSTRACT

The instant invention provides silencer expression cassettes for delivery of RNAi therapy. The silencer cassettes of the instant invention encode an RNAi agent and lack means for replication within a host cell, inverted terminal repeats and further lacking one or more selectable markers. Optionally, the silencer cassettes lack CpG dinucleotides and/or the RNAi agent is within a scaffold of a naturally occurring miRNA. The composition comprising the silencer cassettes of the instant invention, as well as methods of use and suitable systems are also provided.

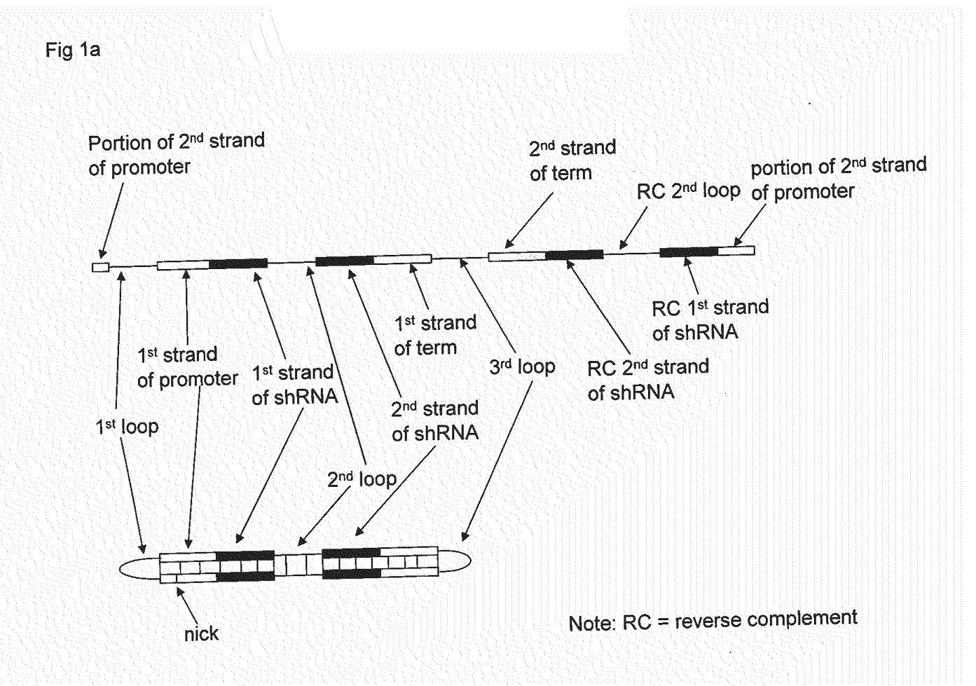

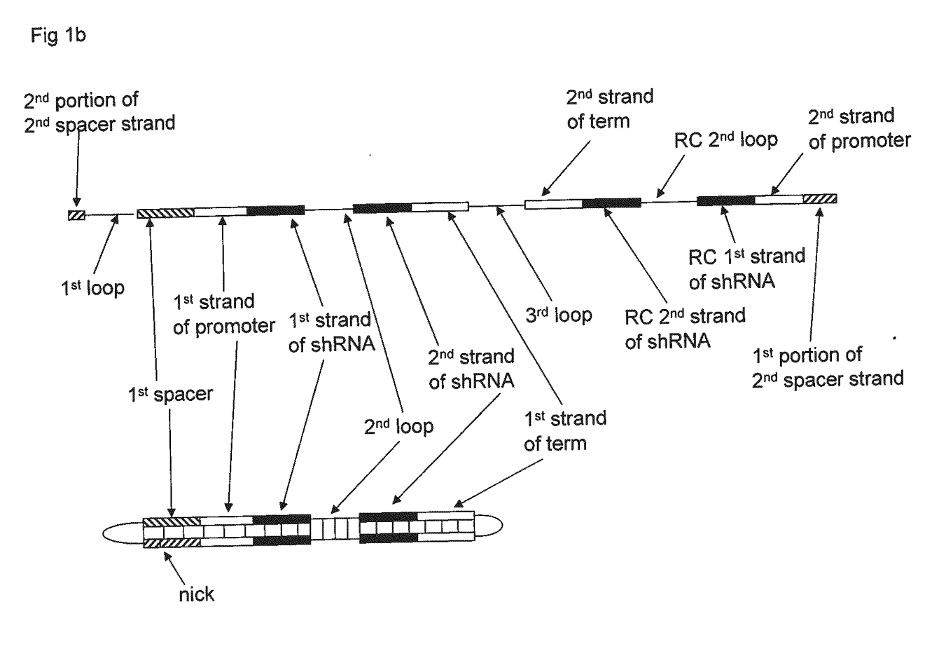

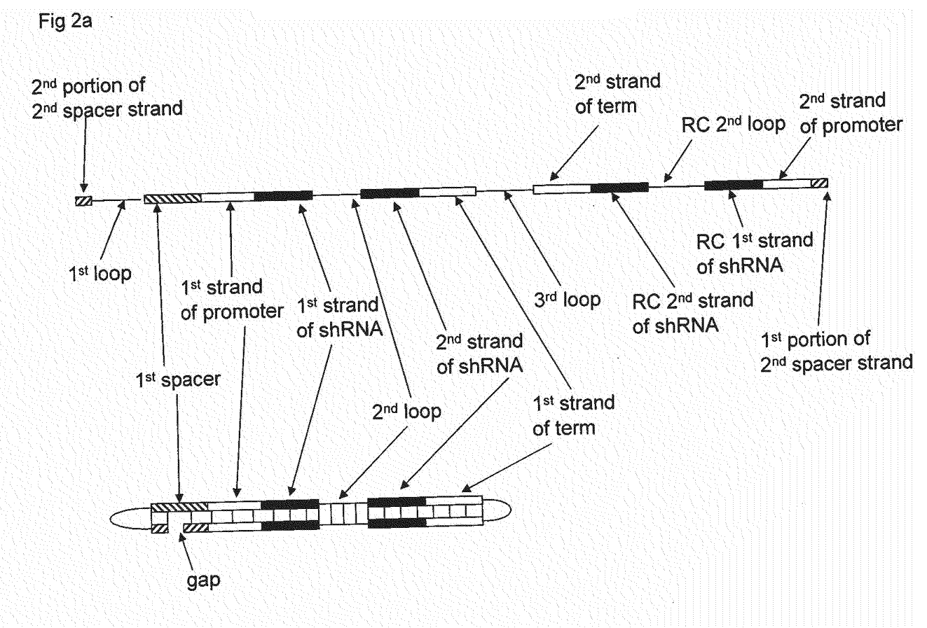

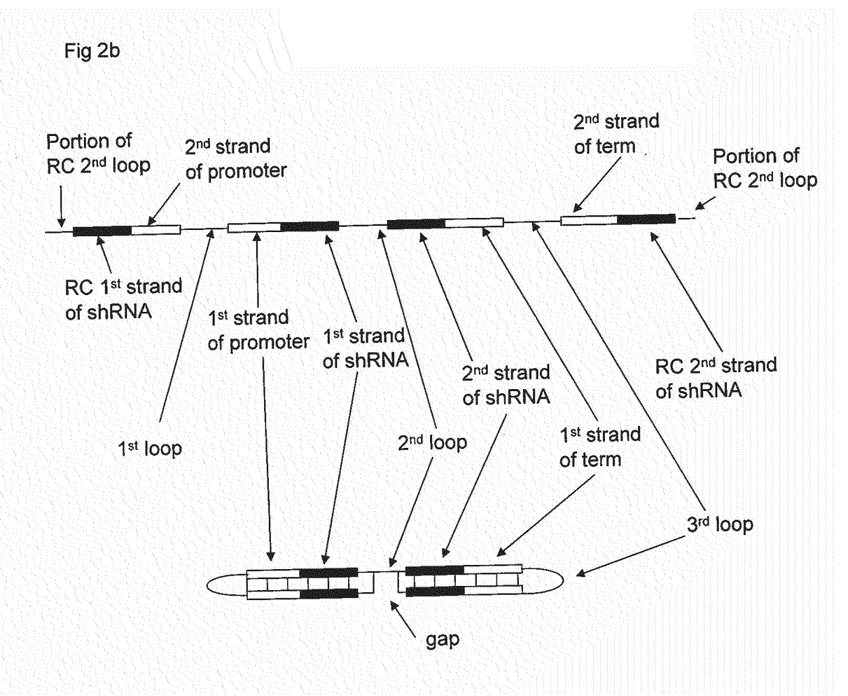

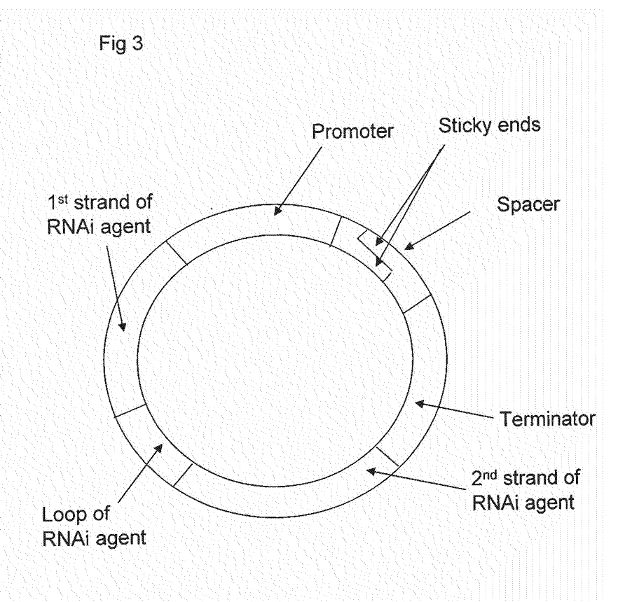

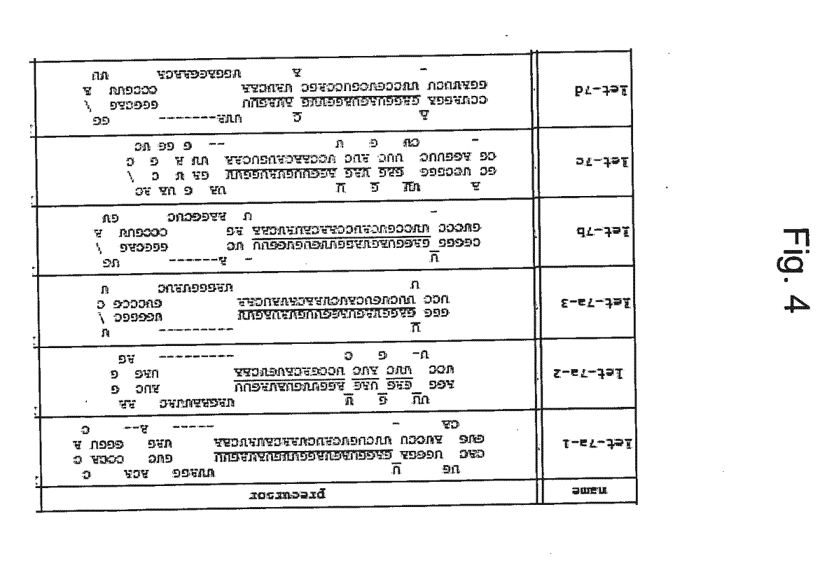

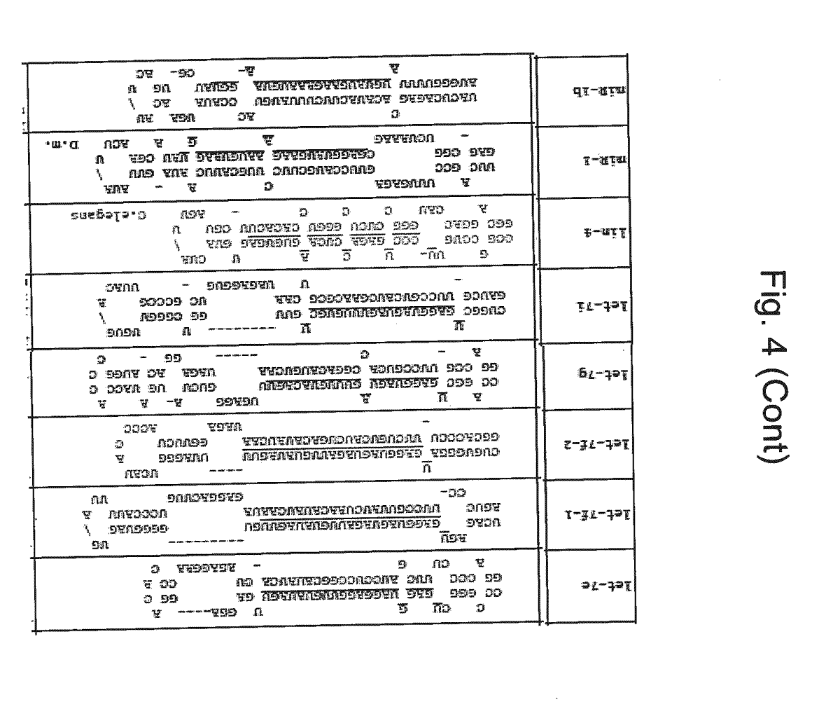

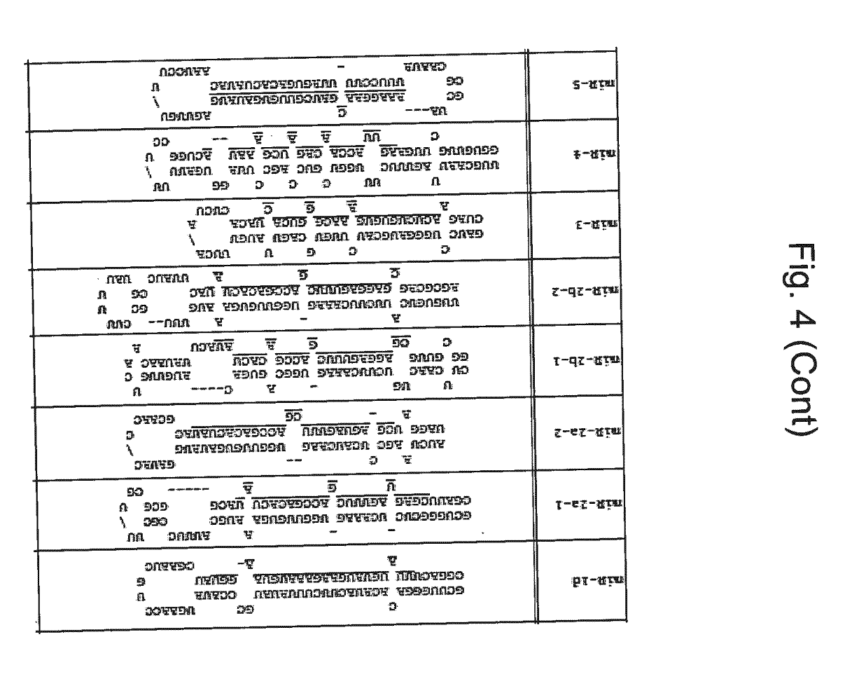

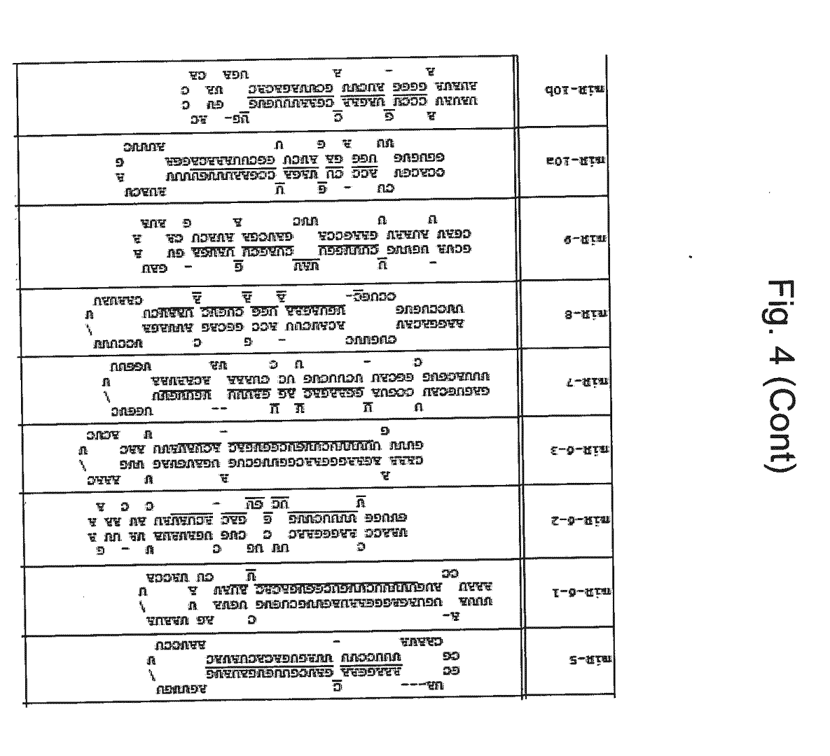

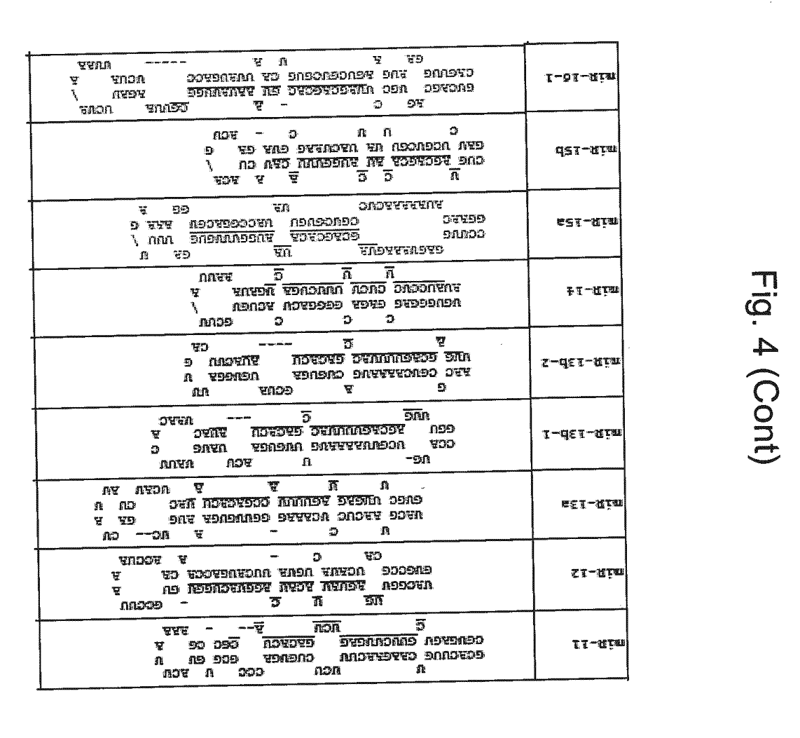

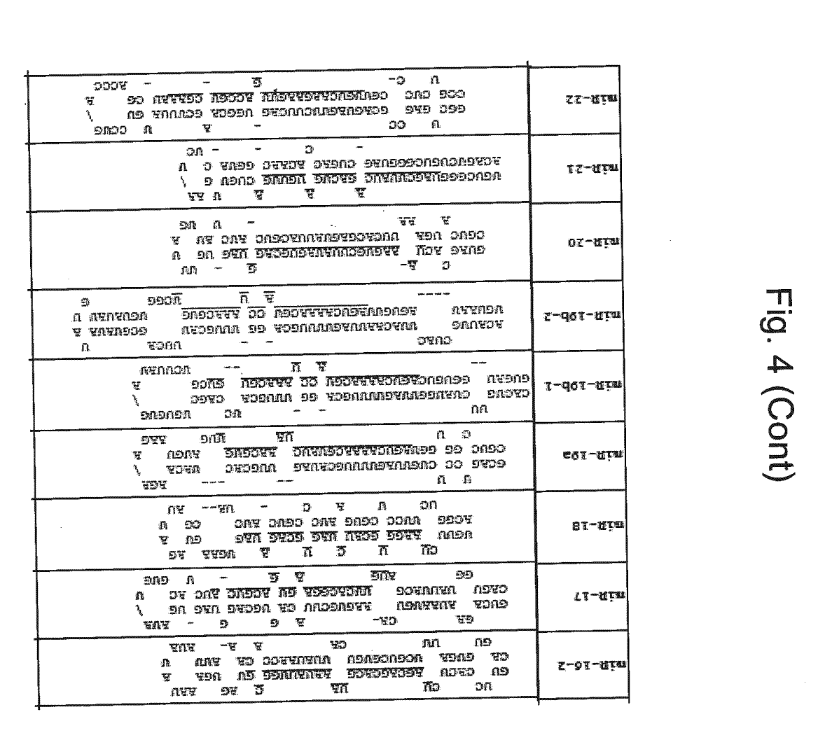

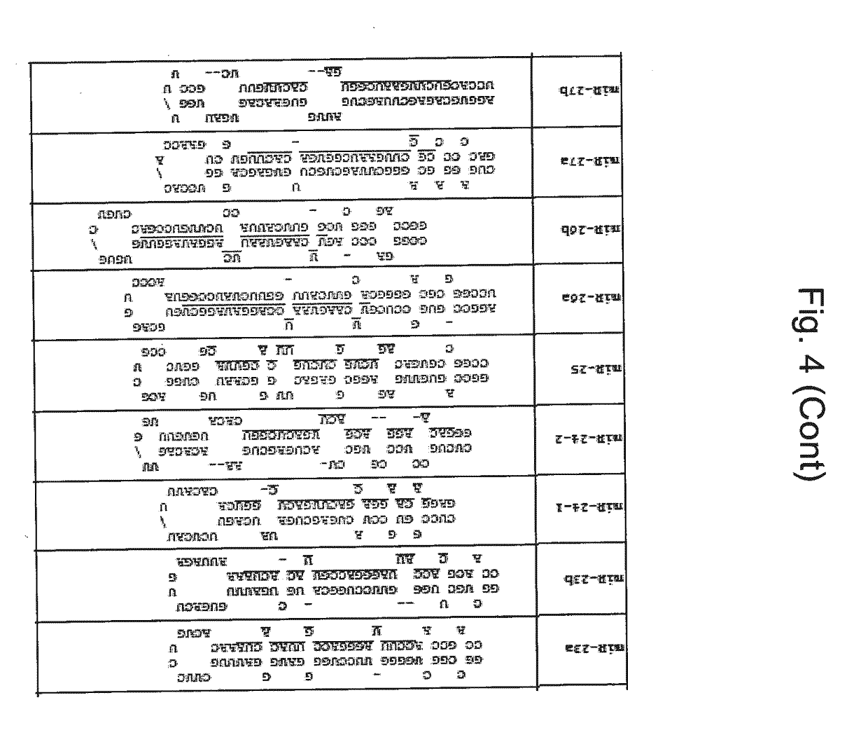

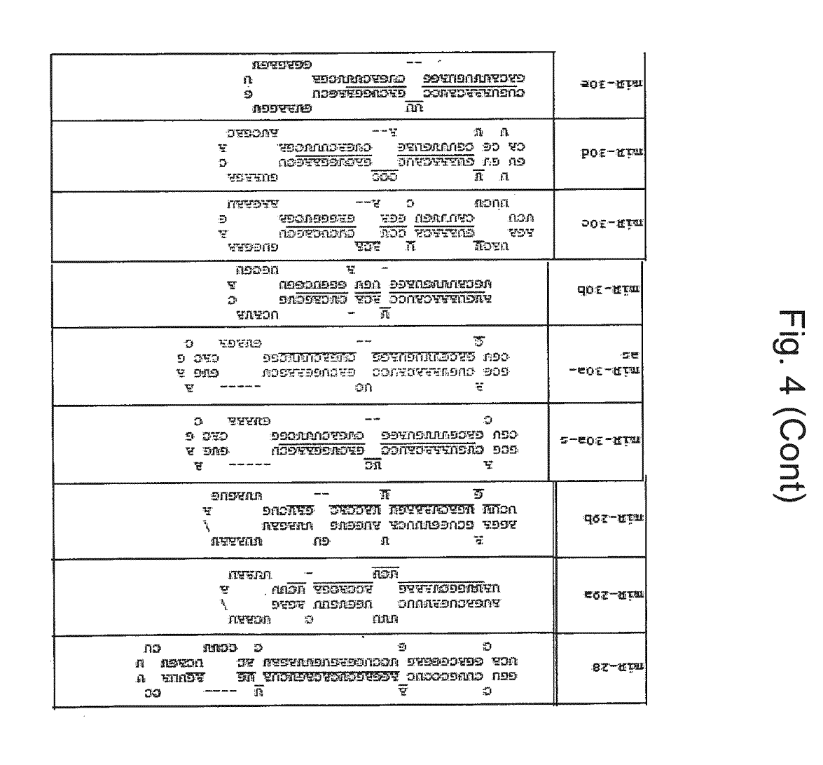

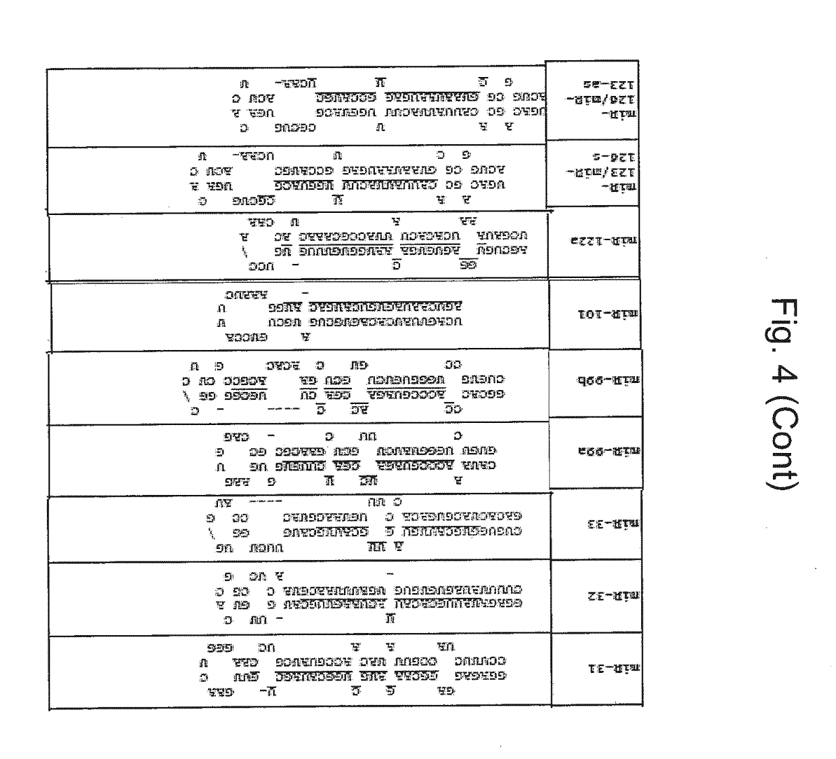

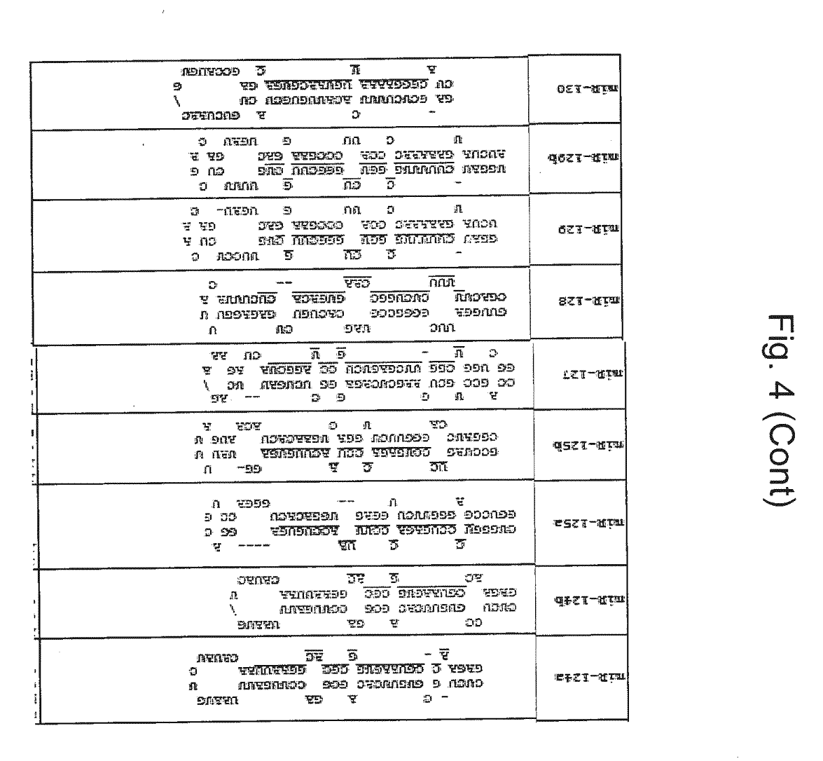

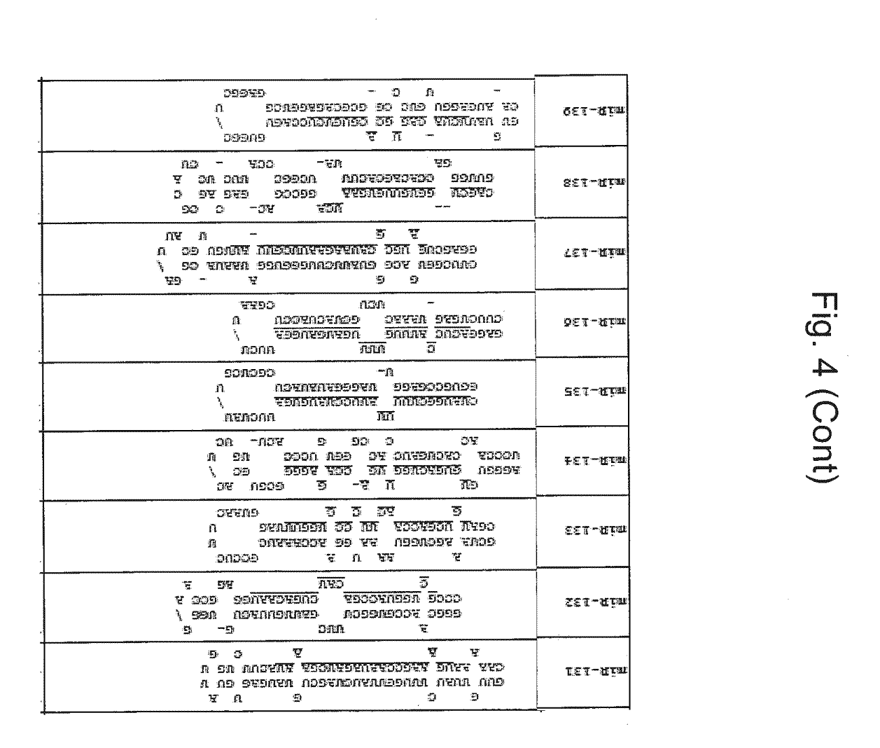

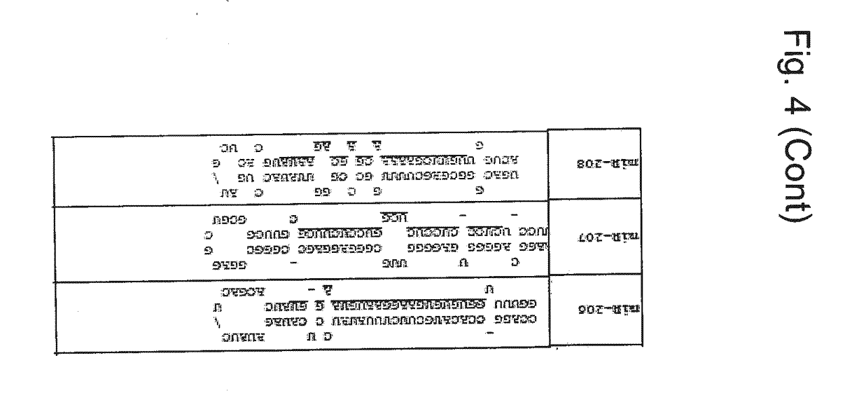

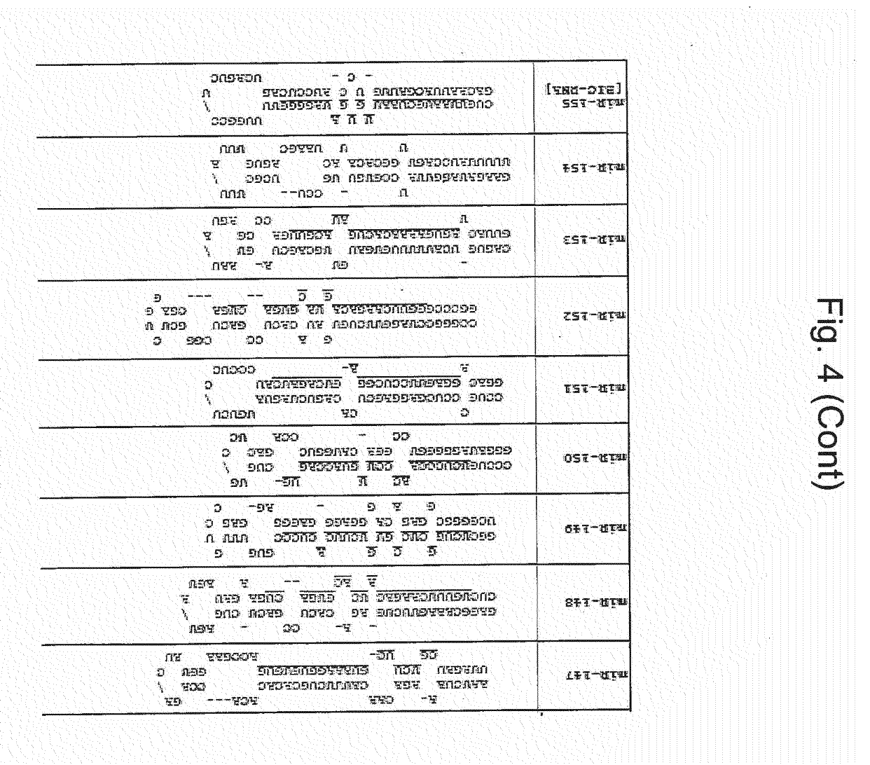

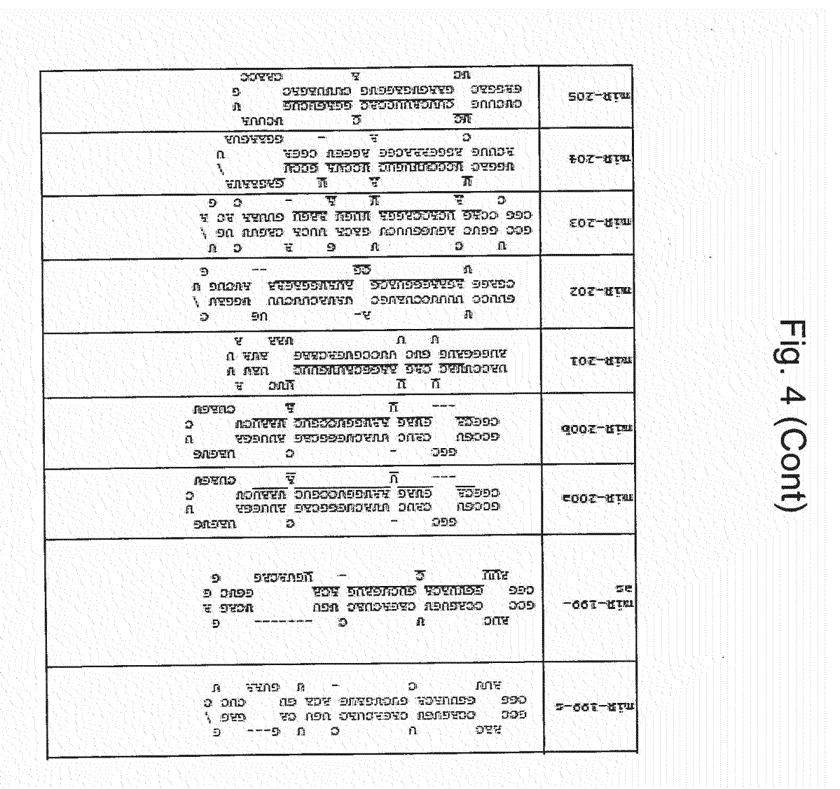

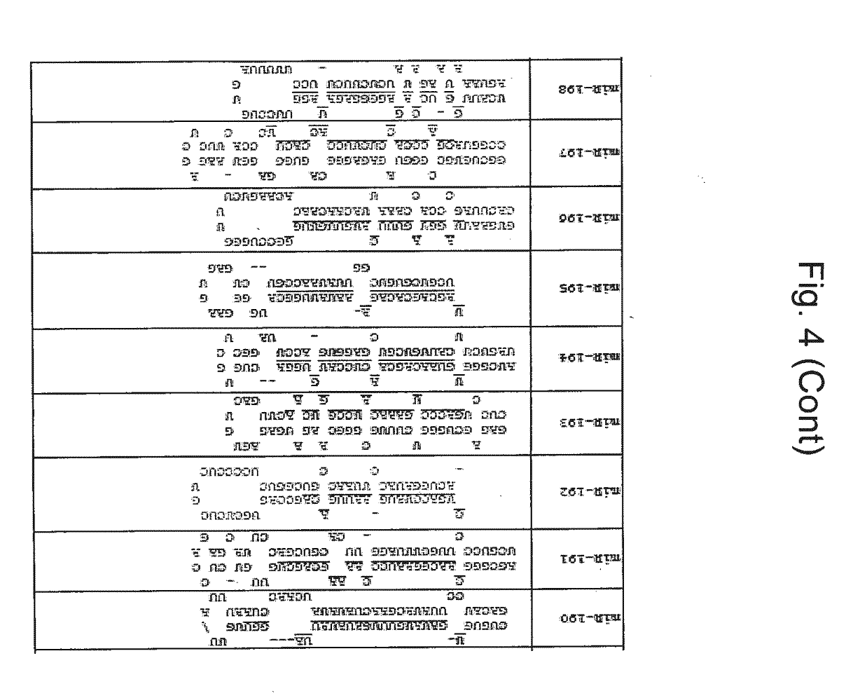

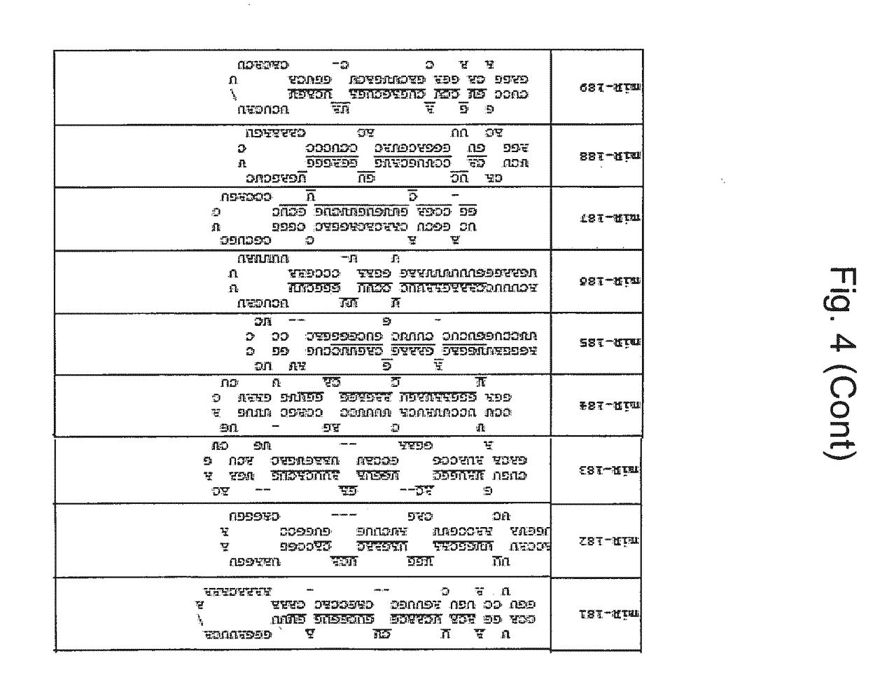

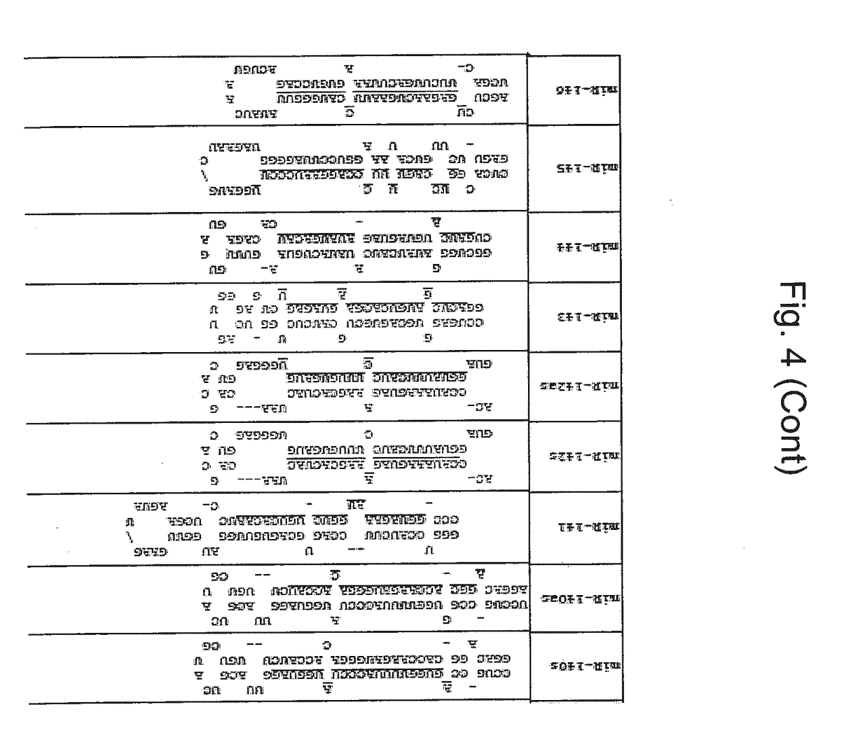

### FIELD OF INVENTION

The instant invention relates to compounds, compositions, and systems for improved delivery of siRNA therapies.

### BACKGROUND

The design and use of small interfering RNA complementary to mRNA targets that produce particular proteins is a recent tool employed by molecular biologists to prevent translation of specific mRNAs. Various groups have been recently studying the effectiveness of siRNAs as biologically active agents for suppressing the expression of specific proteins involved in neurological disorders. Caplen, et al. (Human Molecular Genetics, 11 (2): 175-184 (2002)) assessed a variety of different double stranded RNAs for their ability to inhibit cell expression of mRNA transcripts of the human androgen receptor gene containing different CAG repeats. Their work found gene-specific inhibition occurred with double stranded RNAs containing CAG repeats only when flanking sequences to the CAG repeats were present in the double stranded RNAs. They were also able to show that constructed double stranded RNAs were able to rescue caspase-3 activation induced by expression of a protein with an expanded polyglutamine region. Xia, Mao, et al. (Nature Biotechnology, 20: 1006-1010 (2002)) demonstrated the inhibition of polyglutamine (CAG) expression in engineered neural PC12 clonal cell lines that express a fused polyglutamine-fluorescent protein using constructed recombinant adenovirus expressing siRNAs targeting the mRNA encoding green fluorescent protein.

Small RNA molecules delivered systemically will not persist in vivo long enough to reach the desired target, nor are they likely to cross the blood-brain barrier. Further, the approach taken by the prior art may be impractical because of the large quantity of small interfering RNA that might have to be administered by this method to achieve an effective quantity in the brain.

One major factor that inhibits the effect of siRNAs is the degradation of siRNAs by nucleases. A 3′-exonuclease is the primary nuclease activity present in serum and modification of the 3′-ends of antisense DNA oligonucleotides is crucial to prevent degradation (Eder et al., 1991). An RNase-T family nuclease has been identified called ERI-1 which has 3′ to 5′ exonuclease activity that is involved in regulation and degradation of siRNAs. 5′ to 3′ exonucleases are also known to exist.

RNase A is a major endonuclease activity in mammals that degrades RNAs. It is specific for ssRNA and cleaves at the 3′-end of pyrimidine bases. SiRNA degradation products consistent with RNase A cleavage can be detected by mass spectrometry after incubation in serum (Turner et al., 2007). The 3′-overhangs enhance the susceptibility of siRNAs to RNase degradation. Depletion of RNase A from serum reduces degradation of siRNAs; this degradation does show some sequence preference and is worse for sequences having poly A/U sequence on the ends. Two approaches have been used to overcome this problem. First, the art attempted to use vectors, such as viral vectors. Second, inclusion of modified nucleotides has been contemplated to create bonds which are resistant to exonuclease activity.

An approach of using viral vectors to deliver genes or gene suppressing agents to the brain tissue using stereotactic neurosurgery including, for example, the use of adeno-associated virus (AAV) to deliver gene therapy to the subthalamic nucleus, has shown considerable promise. However, the usefulness of stereotactic neurosurgery to deliver a viral vector carrying a gene or protein suppression therapy can be limited by one or more of the following factors.

Manufacturing of viral particles (e.g., capsid plus DNA payload) in sufficient quantities for therapeutic use, while feasible, is costly relative to production of DNA alone. Viral particles (i.e., the capsid proteins) might be immunogenic, causing adverse reactions in sensitized individuals. While the immune response to some viruses (e.g., AAV) when administered to the brain appears minimal, it remains a potential limitation particularly for repeated therapy administrations.

Further, synthetic nucleic acids introduced into cells or live animals can be recognized as “foreign” and trigger an immune response. Immune stimulation constitutes a major class of off-target effects which can dramatically change experimental results and even lead to cell death.

Accordingly, further compositions, systems and methods are needed for improved delivery of RNAi agents.

### SUMMARY OF INVENTION

The instant invention addresses the deficiencies of the prior art by providing, in one aspect, a silencer cassette in a dumbbell shape, said cassette encoding an RNAi agent and lacking means for replication within a host cell, inverted terminal repeats and further lacking one or more selectable markers. The cassette may comprise double-stranded portion flanked by a first loop and a third loop, wherein the double-stranded portion comprises a promoter sequence located upstream of a sequence encoding a first strand of the RNAi agent located upstream of a sequence encoding a second loop, the second loop being a loop of the RNAi agent, the sequence encoding the second loop located upstream of a sequence encoding a second strand of the RNAi agent located upstream of a terminator sequence and wherein the first loop connects a first strand and a second strand of the promoter sequence, and the third loop connects a first strand and a second strand of the terminator sequence. The terminal 3′ nucleotide may optionally be ligated to the terminal 5′ nucleotide, thus forming a cassette without any nicks or gaps.

Alternatively, the silencer cassette is a double stranded construct in a circular shape, comprising complementary sticky ends.

In one embodiment, the silencer cassette does not include CpG dinucleotides.

Optionally, in any of the embodiments above, the RNAi agent may be placed in a scaffold of a naturally occurring miRNA.

In a second aspect, the invention also provides a composition comprising the silencer cassette according to any of the embodiments of the first aspect of the invention, and a pharmaceutically acceptable carrier.

In a subset of the embodiments, the silencer cassette is encapsulated into a liposome and/or a nanocontainer. Some of the embodiments entailing liposomes and/or nanocontainers are particularly advantageous for administering the composition across the blood brain barrier.

In a third aspect, the invention provides methods of treating a patient by administering the compositions of the second aspect of the invention to a patient.

In a fourth aspect, the invention provides medical systems suitable for administering the compositions of the instant invention to a patient. In one subset of embodiments, the medical system comprises medical system for treating a neurodegenerative disorder of a patient comprising an intracranial access device, a mapping means for locating a predetermined target region in the brain of the patient, a deliverable amount of the pharmaceutical composition comprising the silencer cassette of the instant invention, and a delivery means for delivering said pharmaceutical composition to the predetermined target region in the brain of said patient from said intracranial access device through a stereotactically positioned catheter, said catheter comprising a radiographic marker.

Preferably, the mapping means for locating a predetermined target region in the brain of the patient are intraoperative (e.g., image guided) and/or patient/specific.

In another set of embodiments, the medical system of the instant invention is particularly suitable for delivery of the therapy across blood brain barrier. One such medical system comprises a neurovascular catheter having a distal end positioned in a blood vessel supplying a patient's brain and a means for delivering to the catheter a composition, the means comprising the pharmaceutical composition comprising the cassette of the instant invention encapsulated within a nanocontainer or a liposome, as discussed above, and a component to deliver at least the silencer cassette across the blood-brain barrier.

### DETAILED DESCRIPTION OF PREFERRED EMBODIMENTS

For the purposes of better understanding of the instant disclosure, the following non-limiting definitions have been provided:

The term “about” will be understood by persons of ordinary skill in the art and will vary to some extent according to the context in which it is used. If there are uses of the term which are not clear to persons of ordinary skill in the art given the context in which it is used, “about” will mean up to plus or minus 10% of the particular term.

The term “chronically implanted” with respect to a device refers to a device that remains in the body of a patient, after being positioned in a bodily tissue of the patient by a practitioner, for any period of time after the patient encounter with the practitioner is completed and the patient has departed from the presence of the practitioner.

The term “treating” or “treatment” of a disease refers to executing a protocol, which may include administering one or more drugs to a patient (human or otherwise), in an effort to alleviate signs or symptoms of the disease. Alleviation can occur prior to signs or symptoms of the disease appearing, as well as after their appearance. Thus, “treating” or “treatment” includes “preventing” or “prevention” of disease. In addition, “treating” or “treatment” does not require complete alleviation of signs or symptoms, does not require a cure, and specifically includes protocols which have only a marginal effect on the patient.

The term “patient” refers to a biological system to which a treatment can be administered. A biological system can include, for example, an individual cell, a set of cells (e.g., a cell culture), an organ, a tissue, or a multi-cellular organism. A “patient” can refer to a human patient or a non-human patient.

The term “practitioner” refers to a person who uses methods, systems and compositions of the current invention on the patient. The term includes, without limitations, doctors, nurses, scientists, and other medical or scientific personnel.

By “small interfering RNA” or “siRNA” is meant a nucleic acid molecule which has complementarity in a substrate binding region to a specified gene target, and which acts to specifically guide enzymes in the host cell to cleave the target RNA. That is, the small interfering RNA by virtue of the specificity of its sequence and its homology to the RNA target, is able to cause cleavage of the RNA strand and thereby inactivate a target RNA molecule because it is no longer able to be transcribed. These complementary regions allow sufficient hybridization of the small interfering RNA to the target RNA and thus permit cleavage. One hundred percent complementarity often necessary for biological activity and therefore is preferred, but complementarity as low as 90% may also be useful in this invention. The specific small interfering RNA described in the present application are not meant to be limiting and those skilled in the art will recognize that all that is important in a small interfering RNA of this invention is that it have a specific substrate binding site which is complementary to one or more of the target nucleic acid regions.

Small interfering RNAs are double stranded RNA agents that have complementary to (i.e., able to base-pair with) a portion of the target RNA (generally messenger RNA). Generally, such complementarity is 100%, but can be less if desired, such as 91%, 92%, 93%, 94%, 95%, 96%, 97%, 98%, or 99%. For example, 19 bases out of 21 bases may be base-paired. In some instances, where selection between various allelic variants is desired, 100% complementary to the target gene is required in order to effectively discern the target sequence from the other allelic sequence. When selecting between allelic targets, choice of length is also an important factor because it is the other factor involved in the percent complementary and the ability to differentiate between allelic differences.

By the term “inhibit” or “inhibitory” it is meant that the activity of the target genes or level of mRNAs or equivalent RNAs encoding target genes is reduced below that observed in the absence of the provided small interfering RNA. Preferably the inhibition is at least 10% less, 25% less, 50% less, or 75% less, 85% less, or 95% less than in the absence of the small interfering RNA.

The methods of the present invention utilize routine techniques in the field of molecular biology. Basic texts disclosing general molecular biology methods include Sambrook et al., Molecular Cloning, A Laboratory Manual (3d ed. 2001) and Ausubel et al., Current Protocols in Molecular Biology (1994). Expression of a short hairpin RNA (shRNA) molecule from a double-stranded silencer expression cassette (SEC) is likely prone to degradation by exonucleases. Removal of 5′ and 3′ DNA termini from the therapeutic molecule potentially would stabilize the SEC and permit long term expression of the therapeutic. One means to do is to create a DNA dumbbell molecule containing non-homologous loops on both ends of the SEC. This approach potentially will temper the level of expression of the therapeutic molecule, mitigating any toxicity, yet maintaining its efficacy.

In one embodiment, the silencer cassette of the instant invention comprises a nucleic acid construct, wherein the 5′ and the 3′ ends are protected from exonucleases. The cassette according to the invention, lacks genetic material necessary for self-replication (such as, for example, an on region) or inverted terminal repeats common to viral vectors. In a preferred embodiment, the cassette does not include any selection markers.

In one embodiment, the cassette is essentially a molecule forming a stem, which may or may not be double-stranded, the stem portion flanked by loops on each side of the stem.

The cassette in certain embodiments comprises a double-stranded portion flanked by a first loop and a third loop, wherein the double-stranded portion comprises a promoter sequence located upstream of a sequence encoding a first strand of the RNAi agent located upstream of a sequence encoding a second loop, the second loop being a loop of the RNAi agent, the sequence encoding the second loop located upstream of a sequence encoding a second strand of the RNAi agent located upstream of a terminator sequence. Thus, the first loop connects a first strand and a second strand of the promoter sequence, and the third loop connects a first strand and a second strand of the terminator sequence.

Once the cassette is prepared and placed in the appropriate conditions (e.g., the appropriate temperature and the ionic strength of the solution), the cassette will fold on itself thereby forming a double-stranded stem portion, flanked by two single-stranded loops. The double stranded portion would comprise the promoter, the RNAi agent (including two strands separated by as loop), and the terminator. Within this double-stranded stem portion, one strand is contiguous, and the other strand may comprise a nick or a gap. In some embodiments, it may be beneficial to remove the nick by ligating the nucleotides on the opposite sides of the nick. In other embodiments, where the practitioner decides to keep the nick, preferably, the nick or the gap are located within portions which do not influence the expression of the RNAi agent. To further assure that the nick or the gap do not have such an effect, a spacer may be present in the cassette. Preferred location of the spacer is upstream of the promoter and downstream of the terminator. The distance between the closest flanking loop and the nick should be sufficient to keep the cassette folded on itself in the physiological conditions, present within the patient, including the conditions in the intracellular environment, intercellular space and/or blood.

In another embodiment, the cassette of the instant invention is a double-stranded molecule and does not include single-stranded flanking loops. An exemplary embodiment of such a cassette comprises a promoter, an RNAi agent, a terminator, and two flanking sequences located, respectively, upstream of the promoter and downstream of the terminator. These flanking sequences comprise sticky ends, which, advantageously may be obtained after endonuclease digestion. Thus, in one embodiment, each of the flanking sequences comprises a site for an endonuclease digestion. When the cassette folds on itself, the sticky ends form complementary A-T and C-G bonds, thus holding the cassette together and protecting the ends from the endonuclease digestion.

In another embodiment, the cassette in its final form does not include the site for the endonuclease digestion. Instead, at the manufacturing stage, the cassette comprises a site for a IIs restriction endonuclease. Suitable non-limiting example of such an endonuclease is FokI, which leaves 4-nucleotide-long sticky ends. Other IIs endonucleases also exist and are available from commercial suppliers, such as, for example, New England Biolabs (Ipswich, Mass.). It should be understood that enzymes producing longer sticky ends are preferred to the enzymes producing blunt ends or shorter sticky ends (e.g., 1 nucleotide). The strandedness of the sticky ends (i.e., whether the sticky end is on a 3′ or a 5′ end of a strand) is not important.

In this embodiment, the flanking sequences may be designed in such a way as to minimize the lengths of the respective flanking sequences and to allow for a specific design of the sticky ends.

Another phenomenon interfering with expression of genetic material transferred into a cell (e.g., transgene or siRNA) is methylation. It has been known for a number of years that the presence of methyl (—CH₃) groups attached to DNA is a common feature among eukaryotic organisms. Specifically, the methyl group appears to be preferentially associated with cytosine bases, and the modified base is called 5′-methylcytosine (5′-mC). In general, it has been demonstrated experimentally that there is an inverse relationship between the levels of methylation and gene expression. Methylation generally occurs on cytosine residues which are followed by guanine residue, as may be illustrated by a sequence CpG. Between 60-90% of all CpGs are methylated in mammals. Tucker, Neuron. 30 (3): 649-52 (2001). CpG islands are often located around the promoters of housekeeping genes or other genes frequently expressed in a cell. At these locations, the CG sequence is not methylated. By contrast, the CG sequences in inactive genes are usually methylated to suppress their expression.

Methylation of a transcribed portion of DNA is also known. For example, Kim et al report methylation of exon 1 of TCF-4, associated with the decreased expression of that protein. Carcinogenesis 29 (8): 1623-1631 (2008). Similarly, Glenn et al mention methylation of exon 1 of maternal (non-transcribed) allele of SNRPN gene, and lack of methylation at the same site of the paternal allele. Am. J. Hum. Genet. 58: 335-346 (1996).

However, the removal of CpG dinucleotides in RNAi agents is not a straightforward solution of the problem of achieving a stable expression of the RNAi agent. Galitskii discloses that 5′-CG-3′ dinucleotide and 5′-CNG-3′ trinucleotide are found in published sequences of small interfering RNA and microRNA more often than they should be found in a random sequence and speculates that this is evidence of an important biological purpose of 5′-CG-3′ dinucleotides and 5′-CNG-3′ trinucleotides in small RNA sequences. The author further suggests that the siRNA or miRNA functions not only to guide the RISC complex to catalytically cleave a matching mRNA, but also to bind to DNA and promote the methylation of DNA.

Without wishing to be bound by any theory, for the purpose of longevity of expression of siRNA from those (artificial) cassettes (transduced into cells) the inventors propose that the expression cassette according to any of the embodiments described above may should be designed to lack CpG dinucleotides which may be methylated. Methylation of CpG dinucleotides generally results in lower level of expression of the methylated gene.

It has been previously shown by McBride et al that the administration of an anti-htt siRNA to a mouse striatum may cause neurotoxicity which may or may not be related to the expression of huntingtin. In contrast, when placed into a context of a naturally occurring miRNA, the siRNA was still capable of an efficient inhibition of huntingtin by without associated neurotoxicity. See Proc. Natl. Acad. Sci. USA 105 (15): 5868-5873 (2008). Accordingly, in another set of embodiments, which may be combined with any embodiments described above, the siRNA is placed within a scaffold of a naturally occurring miRNA. The reference to the siRNA placed into “a scaffold of a naturally occurring miRNA” describes a nucleic acid sequence encoding a naturally occurring miRNA, wherein the biologically active portion(s) of the naturally occurring miRNA involved in recruitment of the RISC complex have been replaced with the corresponding strands of a siRNA.

Many miRNAs are conserved in sequence between distantly related organisms, and exhibit tissue-specific or developmental stage-specific expression. The conservation of the sequence between organisms indicates that miRNAs may play important roles in biological processes.

Sequences of miRNAs suitable for the instant invention have been published and are available from public databases, such as, for example, BLAST (see, e.g., Altschul et al. J. Mol. Biol., 215, 403-410 (1990)) or a database from Wellcome Trust of Sanger Institute (Manchester, UK). The web site for this database is featured in Science 303:1741 (2004) and Nature Reviews Genetics 5:244 (2004). See also Griffiths-Jones et al., Nucl. Ac. Res. 2006 34 (Database Issue):D140-D144.

Preferably, the miRNA scaffold selected based on the species of a patient and the target cell type. For example, if the silencer cassette is administered to neurons of a human patient, then the preferred scaffold source is a miRNA which is expressed in human neurons.

As noted above, in the embodiments of the instant invention, the silencer expression cassettes comprise a promoter and a terminator. A variety of suitable promoters and terminators is available in the art. Preferably, the promoter is a pol III promoter. Pol III promoters include the U6 promoter, tRNA promoter, retroviral LTR promoter, Adenovirus VA1 promoter, 5Sr RNA promoter, 7SK RNA promoter, 7SL RNA promoter, and H1 RNA promoter. The U6 promoter adds four uridine nucleotides to the 3′ end of RNA, thus the 3′ overhang of the finally produced siRNA can be freely made to be of 4, 3, 2, 1, or 0 nucleotide by providing the 5′ end sequence of the antisense and sense code DNAs with 0, 1, 2, 3 or 4 adenines. In the case of using other promoters, the number of 3′ overhanging nucleotide can be freely altered. The terminator is also preferably a pol III terminator (TTTTTT, SEQ ID NO: 1).

The silencer expression cassettes according to any embodiments of the instant invention may be prepared according to routine methods in molecular biology, including, without limitations, different PCR techniques, cloning, endonuclease digestion, ligation, etc.

Further, the silencer cassettes according to any embodiment described above may be prepared by using the methods described, e.g., in US Publication 20030054392. This method is particularly suitable for the embodiments which contain spacers. Briefly, a double-stranded stem portion is prepared, comprising sticky ends at each side, and ligated with loop-stem single-stranded nucleotides, having overhangs at the stem portions, said overhangs complementary to the sticky ends of the double-stranded stem portion of the cassette.

In another embodiment, the silencer cassette cloned into a vector may be amplified using a pair of primers only one of each is labeled, e.g., with biotin. Thus, in the double-stranded PCR product, only one strand would be labeled. This strand may be captured in the conditions which both promote melting of the double-stranded PCR product and do not inhibit binding between the label and a capturing substance. In case of biotin, the capturing substance may advantageously be avidin. Thus a single-stranded silencer cassette will be obtained. This cassette may further be re-folded back on itself in appropriate conditions, thus forming the double-stranded stem portion flanked by two loops.

The silencer expression cassettes of the instant invention may be used to deliver RNAi agents suitable for treatments of multiple disease, most advantageously, neurodegenerative diseases. Non-limiting examples of such diseases are provided in table 1.

TABLE 1
Triplet Repeat Expansion Disorders

| | | | | |
|---|---|---|---|---|
| Disease | Symptoms | Gene | Locus | Protein |

Non-coding repeats

| | | | | |
|---|---|---|---|---|
| Dystrophia myotonica 1 | Weakness, Myotonia | DMPK | 19q13 | Dystrophia myotonica |
| | | | | Protein kinase |
| Spinocerebellar ataxia 8 | Ataxia | Antisense | 13q21 | Undetermined |
| | | to KLHL1 | | |
| Huntington disease- | Chorea, dementia | JPH3 | 16q24.3 | Junctophilin 3 |
| like2 | | | | |

Polyglutamine disorders

| | | | | |
|---|---|---|---|---|
| Spinal and bulbar | Weakness | AR | Xq13-q21 | Androgen receptor |
| muscular atrophy | | | | |
| Huntington disease | Chorea, dementia | IT15 | 4P16.3 | Huntingtin |
| Dentatorubral- | Ataxia, myoclonic | DRPLA | 12p13.31 | Atrophin 1 |
| pallidoluysian atrophy | epilepsy, dementia | | | |
| Spinocerebellar ataxia 1 | Ataxia | SCA1 | 6p23 | Ataxin 1 |
| Spinocerebellar ataxia 2 | Ataxia | SCA2 | 12q24.1 | Ataxin 2 |
| Spinocerebellar ataxia 3 | Ataxia | SCA3/MJD | 14q32.1 | Ataxin 3 |
| (Machado-Joseph disease) | | | | |
| Spinocerebellar ataxia 6 | Ataxia | CACNA1A | 19p13 | α_{1A}-voltage- |
| | | | | dependent calcium |
| | | | | channel subunit |
| Spinocerebellar ataxia 7 | Ataxia | SCA7 | 3p12-p13 | Ataxin 7 |
| Spinocerebellar ataxia 17 | Ataxia | TBP | 6q27 | TATA box |
| | | | | binding protein |

Polyalanine disorders*

| | | | | |
|---|---|---|---|---|
| Oculopharyngeal dystrophy | Weakenss | PABPN1 | 14q11.2-q13 | Poly (A) - |
| | | | | binding protein 2 |
| Congential central | Respiratory difficulties | PHOX2B | 4p12 | Paired-like |
| hypoventilation syndrome | | | | homeobox 2B |
| Infantile spasms | Mental retardation, | ARX | Xp22.13 | Aristaless- |
| | epilepsy | | | related homeobox, |
| | | | | X-linked |
| Synpolydactyly | Limb malformation | HOXD13 | 2q31-q32 | Homeobox D13 |

*Polyalanine expansions have also been reported among mutations in other genes, including RUNX2 (runt-related transcription factor2) in cleidocranial dysplasia, ZIC2 (Zic family member 2) in holoprosencephaly HOXA13 (homeobox A13) in hand-foot-genital syndrome, and FOXL2 (forkhead box L2) in type II blepharophimosis, ptosis, and epicanthus inversus syndrome. Small aspartic acid repeat expansions have been reported among other mutations in the COMP (cartilage oligomeric mat4rix protein) gene in patients with multiple epiphyseal dysplasia.

Suitable non-limiting examples of siRNAs are recited in table 2.

In another aspect the instant invention provides compositions comprising the silencer cassettes according to any embodiments of the instant invention and pharmaceutically suitable carriers. Those of skill in the art are familiar with the principles and procedures discussed in widely known and available sources as Remington's Pharmaceutical Science (17th Ed., Mack Publishing Co., Easton, Pa., 1985) and Goodman and Gilman's The Pharmaceutical Basis of Therapeutics (8th Ed., Pergamon Press, Elmsford, N.Y., 1990) both of which are incorporated herein by reference.

The choice of the carriers comprising the compositions ultimately depends on the selected route of delivery, such as, intraparenchymal, intraventricular, intrathecal, transvascular, etc). In a preferred embodiment of the present invention, the composition comprising the cassette of the instant invention is formulated in accordance with standard procedure as a pharmaceutical composition adapted for delivered administration to human beings and other mammals. Typically, compositions for intravenous administration are solutions in sterile isotonic aqueous buffer

For example, if the preferred route is an intraparenchymal delivery, the carrier may be water or a physiologically acceptable salt solution. Where necessary, the composition may also include a solubilizing agent and a local anesthetic to ameliorate any pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent.

In cases other than intravenous administration, the composition can contain minor amounts of wetting or emulsifying agents, or pH buffering agents. The composition can be a liquid solution, suspension, emulsion, gel, polymer, or sustained release formulation. The composition can be formulated with traditional binders and carriers, as would be known in the art. Formulations can include standard carriers such as pharmaceutical grades of mannitol, lactose, starch, magnesium stearate, sodium saccharide, cellulose, magnesium carbonate, etc., inert carriers having well established functionality in the manufacture of pharmaceuticals. Various delivery systems are known and can be used to administer a therapeutic of the present invention including encapsulation in liposomes, microparticles, microcapsules and the like.

If the route of delivery is transvascular and if the composition is delivered to the brain of a subject, it can be dispensed with an infusion bottle containing sterile pharmaceutical grade water or saline. Where the composition is administered by injection, an ampule of sterile water for injection or saline can be provided so that the ingredients may be mixed prior to administration.

The compositions of the instant invention may also comprise a component to deliver at least the silencer cassette across the blood-brain barrier. For example, the silencer cassette of any embodiment of the instant invention may be encapsulated into a liposome and/or a nanocontainer. In different embodiments of the invention, the nanocontainer or the liposome of the invention comprises an exterior surface an internal compartment, the silencer cassette being enclosed in said internal compartment, one or more blood-brain barrier and brain cell membrane targeting agents, and one or more conjugation agents wherein each targeting agent is connected to the exterior surface of the liposome via at least one of the conjugation agents.

Preferably, the exterior surface of the liposome or the nanocontainer defines a sphere having a diameter of at most 200 nanometers. In additional embodiments, the nanocontainer or the liposome comprises blood-brain barrier targeting agents and/or brain cell membrane targeting agents. In different embodiments, at least 5 and at most 1000 blood-brain barrier or brain cell membrane targeting agents are conjugated to the surface of the liposome or the nanocontainer, preferably, at least 25 and at most 40 bloodbrain barrier or brain cell membrane targeting agents.

The conjugation agents are also known in the art. In selected non-limiting embodiments, the conjugation agents may be polyethylene glycol, sphingomyelin, biotin, streptavidin, organic polymers, or any combinations thereof.

In yet another aspect, the instant invention provides medical systems suitable for delivery of the silencer cassettes and/or the compositions of the instant invention. In one exemplary embodiment, the medical systems of the instant invention include an intracranial access device, a mapping means for locating a predetermined target region in the brain of the patient a deliverable amount of the silencer expression cassette according to any embodiment described above, or a pharmaceutical composition comprising the silencer expression cassette, and a delivery means for delivering said pharmaceutical composition to the predetermined target region in the brain of said patient from said intracranial access device through a stereotactically positioned catheter, said catheter comprising a radiographic marker.

The target area may be located by many methods. For example, for some application, the targeted area may be located by stereotactical or gross anatomical atlases. In other embodiments, when the precise location of the targeted area is crucial, e.g., when the at least partially reversible gene therapy system is delivered into the brain of the patient, other mapping means may be used. Such mapping means include, without limitation, Positron Emission Tomography and Single Photon Emission Computed Tomography (PET and SPECT, respectively), pharmacological Magnetic Resonance Imaging (phMRI), functional MRI (fMRI), and contrast-enhanced computerized tomography (CT) scan.

In another embodiment, Computer-aided atlas-based functional neurosurgery methodology can be used to accurately and precisely inject the deoxyribonucleic acid of the present invention. Such methodologies permit three-dimensional display and real-time manipulation of cerebral structures. Neurosurgical planning with mutually preregistered multiple brain atlases in all three orthogonal orientations is therefore possible and permits increased accuracy of target definition for treatment injection or implantation, reduced time of the surgical procedure by decreasing the number of tracts, and facilitates planning of more sophisticated trajectories. See e.g. Nowinski W. L. et al., Computer-Aided Stereotactic Functional Neurosurgery Enhanced by the Use of the Multiple Brain Atlas Database, IEEE Trans Med Imaging 19 (1); 62-69:2000.

Preferably, the pre-determined target area in the brain of the patient is determined on an individual basis, e.g., by real time image guidance, so that the neurosurgeon will see exactly where the catheter is being placed. Suitable systems exist for this particular embodiment, including, without limitation, STEALTH station developed by Surgical Navigation Technologies, a division of Medtronic. This tool incorporates preoperative images, including MRI, CT, and functional imaging studies into the computers in the operating room. A hand held probe linked to the computer can be used to point anywhere on the patients head or brain, with the corresponding area shown with great accuracy on a computer screen. Thus, there is no need to guess at the relationship between an area on or in the brain, inspected by sight and where that corresponds to the patient's preoperative images. Medtronic NT StealthStation® Treon™, further refines the computerized technologies of multi-dimensional imaging and navigation to enable neurosurgeons to precisely plan, re-plan, and visualize a procedure as it proceeds deep within the brain for treating neurological disorders in a living human patient.

Generally, neurons affected with Huntington's disease reside in striatum, neurons affected with Alzheimer's disease reside in nucleus basalis of Meynart and the cerebral cortex, and neurons affected with Parkinson's disease reside in the substantia nigra. Thus, in different embodiments depending on the disease, the device delivers the therapies according to the methods of the instant invention to nucleus basalis of Meynart and the cerebral cortex, striatum, and/or the substantia nigra.

Delivery means suitable for the use with the systems of the instant invention are well known in the art. In one embodiment of the invention, the delivery means is an infusion pump, which may be, e.g., an electromechanical pump or an osmotic pump.

Examples of the delivery devices within the scope of the present invention include the Model 8506 investigational device (by Medtronic, Inc. of Minneapolis, Minn.), which can be implanted subcutaneously on the cranium, and provides an access port through which therapeutic agents may be delivered to the brain. Delivery occurs through a stereotactically implanted polyurethane catheter. Two models of catheters that can function with the Model 8506 access port include the Model 8770 ventricular catheter by Medtronic, Inc., for delivery to the intracerebral ventricles, which is disclosed in U.S. Pat. No. 6,093,180, incorporated herein by reference, and the IPA1 catheter by Medtronic, Inc., for delivery to the brain tissue itself (i.e., intraparenchymal delivery), disclosed in U.S. Ser. Nos. 09/540,444 and 09/625,751, which are incorporated herein by reference. The latter catheter has multiple outlets on its distal end to deliver the therapeutic agent to multiple sites along the catheter path. In addition to the aforementioned device, the delivery of the small interfering RNA vectors in accordance with the present invention can be accomplished with a wide variety of devices, including but not limited to U.S. Pat. Nos. 5,735,814, 5,814,014, and 6,042,579, all of which are incorporated herein by reference. Using the teachings of the present invention and those of skill in the art will recognize that these and other devices and systems may be suitable for delivery of small interfering RNA vectors for the treatment of neurodegenerative diseases in accordance with the present invention.

In one set of embodiments, the tip of the catheter comprises a radiographic marker. The nature of the radiographic marker ultimately depends on the method used for imaging of the brain. For example, in one embodiment, the marker renders at least a portion of the tip opaque to x-rays, enabling the tip of the catheter to be observed during fluoroscopy or via x-ray to facilitate placement of the catheter. In this embodiment, the radiographic marker may comprise tantalum powder dispersed in a matrix composed of a biocompatible adhesive. Other materials, such as barium or platinum, are also suitable for the radiographic marker of this embodiment.

In another embodiment, the radiographic marker may be composed of a material that is compatible to nuclear magnetic resonance imaging (MRI) to enable the tip of the catheter to be detected during an MRI scan. A preferred material for the radiographic marker in an MRI context is platinum, though barium, tantalum, and similar materials are also suitable. Regardless of whether radiography or MRI is being utilized, the goal of providing a radiographic marker is to enable the operator to accurately detect the precise location of the tip of the catheter to facilitate placement and later verification of the integrity and position of the catheter.

In another set of embodiments, the medical system of the instant invention is particularly suitable for a transvascular delivery of the silencer expression cassette, particularly, for a delivery through the blood brain barrier. In these embodiments, the medical system comprises a neurovascular catheter having a distal end positionable in a blood vessel supplying a patient's brain, and a means for delivering to the catheter a composition comprising the nanocontainer of the liposome as described above. Preferably, the medical system also comprises a component to deliver at least the silencer cassette across the blood-brain barrier.

It is necessary to note at this point that the blood vessel supplying a patient's brain may be located using the same techniques and equipment suitable for mapping the pre-determined location in the brain. In other embodiments, the catheter may be inserted somewhere else in the patient's body (e.g., femoral artery) and moved along the vascular system until it reaches e.g., internal carotid artery. In some embodiments (e.g., in patients with high stroke risk due to fragile blood vessels), moving the catheter further may be undesirable.

Similarly to the systems for intraparenchymal delivery, in the delivery means suitable for the use with the systems for transvascular delivery are also well known in the art and include an infusion pump, which may be, e.g., an electromechanical pump or an osmotic pump. Further, the distal tip of the catheter may optionally comprise a radiographic marker as described above.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Other suitable modifications and adaptations to the methods and applications described herein are suitable and may be made without departing from the scope-of the invention or any embodiment thereof. While the invention has been described in connection with certain embodiments, it is not intended to limit the invention to the particular forms set forth, but on the contrary, it is intended to cover such alternatives, modifications and equivalents as may be included within the spirit and scope of the invention as defined by the following claims.

1. A silencer cassette in a dumbbell shape, said cassette encoding an RNAi agent and lacking means for replication within a host cell, inverted terminal repeats and further lacking one or more selectable markers.

2. The silencer cassette of claim 1, wherein said cassette does not include CpG dinucleotides.

3. A silencer cassette of claim 1 or 2, comprising a double-stranded portion flanked by a first loop and a third loop, wherein the double-stranded portion comprises:
a) a promoter sequence located upstream of
b) a sequence encoding a first strand of the RNAi agent located upstream of
c) a sequence encoding a second loop, the second loop being a loop of the RNAi agent, the sequence encoding the second loop located upstream of
d) a sequence encoding a second strand of the RNAi agent located upstream of
e) a terminator sequence; and
wherein the first loop connects a first strand and a second strand of the promoter sequence, and the third loop connects a first strand and a second strand of the terminator sequence.

4. The silencer of claim 2 or 3, wherein said sequence encoding the first strand of the RNAi agent and said sequence encoding the second strand of the RNAi agent are present in a scaffold of a naturally occurring miRNA.

5. The silencer cassette of claim 3 or 4 wherein the terminal 3′ nucleotide is ligated to the terminal 5′ nucleotide of the cassette, or wherein the cassette further comprises a nick or a gap in between the terminal 3′ nucleotide and the terminal 5′ nucleotide in the double-stranded portion.

6. The silencer cassette of claim 5, wherein the nick is at least seven nucleotides away from the first or the second loop.

7. The silencer cassette of claim 5, wherein the gap is not greater than three nucleotides.

9. The silencer cassette of claim 5 or 6, wherein the nick is in the first or the second strand of the promoter sequence.

10. The silencer cassette of claim 5 or 6, wherein the nick is in the first or the second strand of the terminator sequence.

11. The silencer cassette of claim 5 or 6, wherein the nick is in the first or the second strand of the sequence encoding the second loop.

12. The silencer cassette of claim 5 or 6, wherein the nick is in a strand of the double stranded portion, wherein said strand does not include portions transcribed into the RNAi agent.

13. The silencer cassette of any one of claims 1-4, further comprising a double-stranded spacer region, wherein said region has a first spacer strand which is contiguous, and a second spacer strand which consists of two portions, said two portions are respectively located at the 5′ and the 3′ ends of the cassette such that when the cassette forms the dumbbell structure, the two portions of the second spacer strand are together complementary to the first spacer strand.

14. The silencer cassette of claim 13, wherein the nick or the gap is in the spacer region.

15. The silencer cassette of claim 13 or 14, wherein the first spacer strand is located immediately adjacent to the first loop or to the third loop.

16. The silencer cassette of claim 5 or 7, wherein the gap is in a strand of the double stranded portion, wherein said strand does not contain portions transcribed into the RNAi agent.

17. A silencer cassette in a circular shape, said cassette encoding an RNAi agent and lacking an on region and one or more selectable markers.

18. The silencer cassette of claim 17, said silencer cassette not including CpG dinucleotides.

19. The silencer cassette of claim 17 or 18, comprising a sequence encoding the first strand of the RNAi agent and a sequence encoding a second strand of the RNAi agent, wherein said sequence encoding the first strand of the RNAi agent and said sequence encoding the second strand of the RNAi agent are present in a scaffold of a naturally occurring miRNA.

20. The silencer cassette of any one of claims 17-19, wherein the first strand and the second strand form sticky ends.

21. The silencer cassette of any one of claims 1-16, wherein the RNAi agent has a first strand having length between 19 and 30 nucleotides, and wherein at least 19 contiguous nucleotides are identical to any one of SEQ ID NOs. 2-144.

22. A pharmaceutical composition comprising the silencer cassette of any one of claims 1-21 and a pharmaceutically acceptable carrier.

23. The pharmaceutical composition comprising the silencer cassette of any one of claims 1-21 encapsulated into a liposome and/or a nanocontainer.

24. The pharmaceutical composition of claim 23, wherein the liposome or the nanocontainer comprises
an exterior surface;
an internal compartment, the silencer cassette being enclosed in said internal compartment;
one or more blood-brain barrier and brain cell membrane targeting agents; and
one or more conjugation agents wherein each targeting agent is connected to the exterior surface of the liposome via at least one of the conjugation agents.

25. The pharmaceutical composition of claim 23 or 24, wherein the exterior surface of the liposome or the nanocontainer defines a sphere having a diameter of at most 200 nanometers.

26. The pharmaceutical composition of any one of claims 23-25, wherein at least 5 and at most 1000 blood-brain barrier or brain cell membrane targeting agents are conjugated to the surface of the liposome.

27. The pharmaceutical composition of any one of claims 23-26, wherein at least 25 and at most 40 bloodbrain barrier or brain cell membrane targeting agents are conjugated to the surface of the liposome.

28. The pharmaceutical composition of any one of claims 23-27, wherein the conjugation agent is selected from the group consisting of polyethylene glycol, sphingomyelin, biotin, streptavidin, organic polymers, and combinations thereof

29. A method of treating a mammal suffering from a neurodegenerative disease comprising administering to a mammal the pharmaceutical composition of any one of claims 22-28.

30. The method of claim 29, wherein the composition is administered into the target region of the brain.

31. The method of claim 30, wherein the composition is according to any one of claims 23-28, and wherein the composition is administered into a blood vessel supplying blood to the target region of the brain.

32. A medical system for treating a neurodegenerative disorder of a human patient comprising:
(a) an intracranial access device;
(b) a mapping means for locating a predetermined target region in the brain of the patient;
(c) a deliverable amount of the pharmaceutical composition of claim 22; and
(d) a delivery means for delivering said pharmaceutical composition to the predetermined target region in the brain of said patient from said intracranial access device through a stereotactically positioned catheter, said catheter comprising a radiographic marker.

33. The medical system of claim 32 wherein said intracranial access device is an intracranial access port.

34. The medical system of any one of claims 32 and 33 wherein said delivery means is injection from an external syringe into an intracranial access port.

35. A medical system for delivering DNA across a blood-brain barrier, the system comprising:
a neurovascular catheter having a distal end positioned in a blood vessel supplying a patient's brain; and
a means for delivering to the catheter a composition comprising:
the pharmaceutical composition according to any one of claims 23-28; and
a component to deliver at least the silencer cassette across the blood-brain barrier.

36. The medical system of any one of claims 32-35 wherein said delivery means is an infusion pump.

37. The medical system of any one of claims 32-36, wherein the said infusion pump is an electromechanical pump.

38. The medical system of any one of claims 32-36, wherein the said infusion pump is an osmotic pump.

39. The method of any one of claims 29-31, or the medical system of any one of claims 32-38, wherein the disease is Huntington's disease and the target region of the brain is selected from the caudate, putamen, or striatum.

40. The method of any one of claims 29-31, or the medical system of any one of claims 32-38, wherein the disease is Alzheimer's disease and the target region of the brain is selected from said location selected from the hippocampus, the nucleus basalis of Meynert and the cerebral cortex.

41. The method of any one of claims 29-31, or the medical system of any one of claims 32-38, wherein the disease is Spinocerebellar ataxia-1 and the target region of the brain is selected from dentate nucleus, emboliform nucleus, the globose nucleus, the fastigial nucleus of the cerebellum (collectively the deep cerebellar nuclei), or the cerebellar cortex.
